(19) United States
(12) Patent Application Publication
(10) Pub. No.: US 2007/0019160 A1
(43) Pub. Date: Jan. 25, 2007

(54) RING LIGHT FUNDUS CAMERA

(75) Inventors: Werner Kleen, Wiederrossea (DE); Wolfgang Sperling, Jena Ot Wogau (DE)

Correspondence Address: DARBY & DARBY P.C., P. O. BOX 5257, NEW YORK, NY, 10150-5257, US

(73) Assignee: Carl Zeiss Meditec AG, Jena (DE)

(21) Appl. No.: 11/459,487

(22) Filed: Jul. 24, 2006

Related U.S. Application Data
(60) Provisional application No. 60/702,038, filed on Jul. 22, 2005.

(51) Int. Cl. A61B 3/14

(52) U.S. Cl. 351/206

(57) ABSTRACT

An ophthalmoscope for viewing a fundus of an eye includes an optical lens, a ring light source configured to project light along an illumination path through the optical lens into the eye, a viewing optical system configured to view the fundus through the optical lens along a viewing optical path, wherein a portion of the viewing optical path and a portion of the illuminating optical path share a same optical axis. In addition, a fundus camera includes a viewing optical path, an imaging device, and an illuminating optical path including at least one LED and a pinhole mirror reflecting the at least one LED into the imaging device, wherein at least a portion of the illuminating optical path shares an optical axis with at least a portion of the viewing optical path.

Priority is claimed to U.S. provisional Patent Application No. 60/702,038, filed on Jul. 22, 2005, the entire disclosure of which is incorporated by reference herein.

The present invention relates to an ophthalmological examination instrument for photographing the fundus of the eye of humans and animals. Furthermore, front sections of the eye can be captured.

### BACKGROUND

This ophthalmological examination instrument is also called a fundus camera. The classic structure of a fundus camera consists of a viewing optical path and an illuminating optical path. In the simplest case, the viewing optical path has two lenses. The image scale is essentially determined by the factor of the two focal lengths of the lenses. On the imaging side of the optical system, the fundus of the eye can be photographed or viewed through imaging devices such as solid state cameras or through light-sensitive films or through an eyepiece. The illuminating optical path of a classical fundus camera is complex. It has the objective of allowing light beams to enter the eye to be viewed without interfering with the viewing optical path in this process. It has to be taken into account that only a fraction of the introduced light is reflected for viewing while the rest is completely absorbed. Light from a source is collimated by means of a condenser outside of the axis of the viewing optical path, it traverses several apertures (iris aperture, cornea aperture and lens apertures) until the light from the source is conducted via a pinhole mirror in the direction of the sagittal axis of the patient's eye and it is projected sharply onto the cornea through the ophthalmoscope lens. A drawback is the complicated structure of the entire optical system with its two separate optical paths. Its production is demanding and it is complicated and difficult to align.

### SUMMARY OF THE INVENTION

An object of the present invention is to provide a simple fundus camera that has a special and simple optical path. All reflections such as the cornea reflection and the ophthalmoscope lens reflection are deflected in such directions that they do not interfere with the viewing optical path.

The present invention relates to an ophthalmological examination instrument for photographing the fundus of the eye of humans and animals. Furthermore, front sections of the eye can be captured. The principle for achieving this is based on the fact that the viewing optical path and the illuminating optical path are mainly on the same optical axis and that the illumination is provided through a ring light arrangement.

### BRIEF DESCRIPTION OF THE DRAWINGS

The present invention is described in more detail below with reference to the accompanying drawings, in which:

FIG. 1 shows a first exemplary embodiment of an ophthalmoscope according to the present invention;

FIG. 2 shows a second exemplary embodiment of an ophthalmoscope according to the present invention;

FIG. 3 shows a third exemplary embodiment of an ophthalmoscope according to the present invention;

FIG. 4a shows a first exemplary embodiment of a ring light according to the present invention;

FIG. 4b shows a second exemplary embodiment of a ring light;

FIG. 4c shows a third exemplary embodiment of a ring light

FIG. 4d shows a fourth exemplary embodiment of a ring light

FIG. 4e shows a fifth exemplary embodiment of a ring light

FIG. 5 shows fourth exemplary embodiment of an ophthalmoscope;

FIG. 6 shows a fifth exemplary embodiment of an ophthalmoscope;

FIG. 7 shows a sixth exemplary embodiment of an ophthalmoscope according to the present invention; and

FIG. 8 shows a seventh exemplary embodiment of an ophthalmoscope according to the present invention;

### DETAILED DESCRIPTION

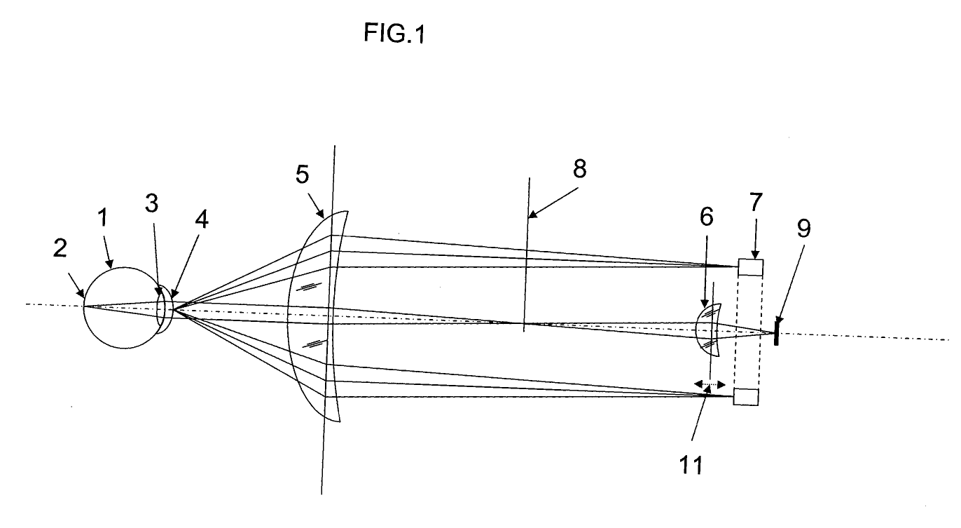

FIG. 1 shows a ophthalmoscope with a solid state camera. The illumination system is the ring light source 7. The viewing optical path includes a solid state surface sensor 9 located in the imaging plane and having a viewing optical system 6 positioned in front of it. The viewing optical path and the illuminating optical path are on one optical axis, the ophthalmoscope lens 5 being shared. The light emitted by the ring light source 7 is assumed to be approximately parallel. The ring light is projected through the ophthalmoscope lens 5 onto the cornea 4 of the patient's eye 1. The ring light projected on the cornea 4 scatters light into the inside of the eye 1. The retina 2 constitutes an illuminated object. The eye lens 3 images the retina 2 into infinity and the ophthalmoscope lens 5 focuses it in an intermediate image plane 8. A viewing optical system 6, which in the simplest case comprises an imaging device including an objective with a solid state surface sensor 9, is needed in order to be able to make the intermediate image visible or to capture it. Any unsharpness in the image is compensated for in the direction of the main optical axis by a fine focusing drive 11 of the viewing optical system 6.

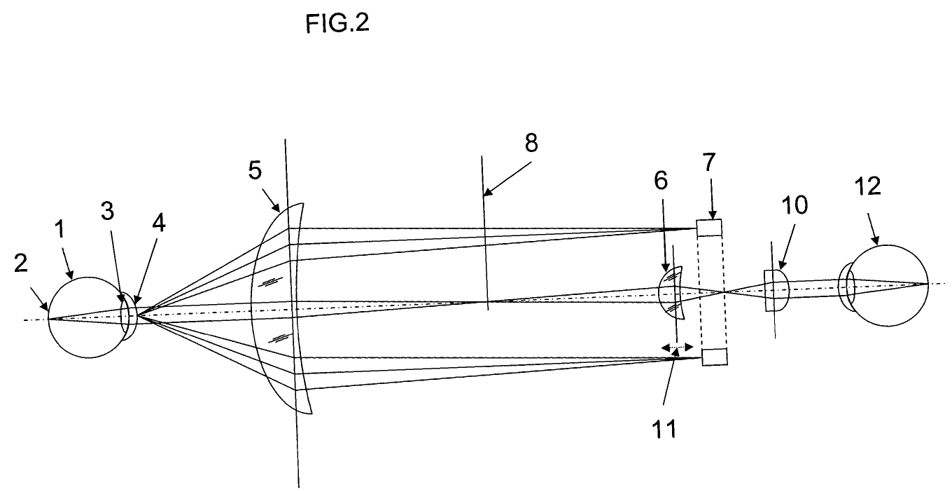

FIG. 2 shows a ophthalmoscope having an eyepiece. The illumination system is the ring light source 7. The viewing optical path includes an eyepiece 10 located in the imaging plane. The viewing optical path and the illuminating optical path are identical, the ophthalmoscope lens 5 being shared. The light emitted by the ring light source 7 is assumed to be approximately parallel. The ring light is projected through the ophthalmoscope lens 5 onto the cornea 4 of the patient's eye 1. The ring light projected on the cornea 4 scatters light into the inside of the eye 1. The retina 2 constitutes an illuminated object. The eye lens 3 images the retina 2 into infinity and the ophthalmoscope lens 5 focuses it in an intermediate image plane 8. The intermediate image becomes visible to the observer through the viewing optical system 6 and through an eyepiece 10. Any unsharpness in the image is compensated for in the direction of the main optical axis by a fine focusing drive 11 of the viewing optical system 6.

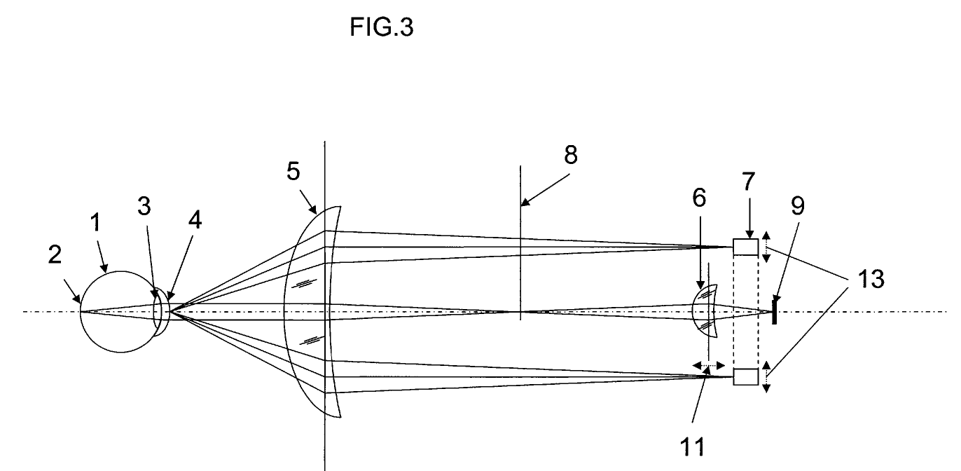

FIG. 3 shows an ophthalmoscope having a variable ring light. The illumination system is the ring light source 7. The viewing optical path includes a solid state surface sensor 9 located in the imaging plane. The viewing optical path and the illuminating optical path are identical, the ophthalmoscope lens 5 being shared. The light emitted by the ring light source 7 is assumed to be approximately parallel. The ring light is projected through the ophthalmoscope lens 5 onto the cornea 4 of the patient's eye 1. The diameter of the ring light 7 is variably adjustable, as a result of which one can set it to the width of the iris. The diameter 13 is set in such a way that, on the one hand, no interfering reflections of the cornea detrimentally affect the image being formed and, on the other hand, the brightness or the contrast of the image being formed are optimal. The ring light projected on the cornea 4 scatters light into the inside of the eye 1. The retina 2 constitutes an illuminated object. The eye lens 3 images the retina 2 into infinity and the ophthalmoscope lens 5 focuses it in an intermediate image plane 8. A viewing optical system, which in the simplest case comprises an objective with a solid state surface sensor 9, is needed in order to be able to make the intermediate image visible or to capture it.

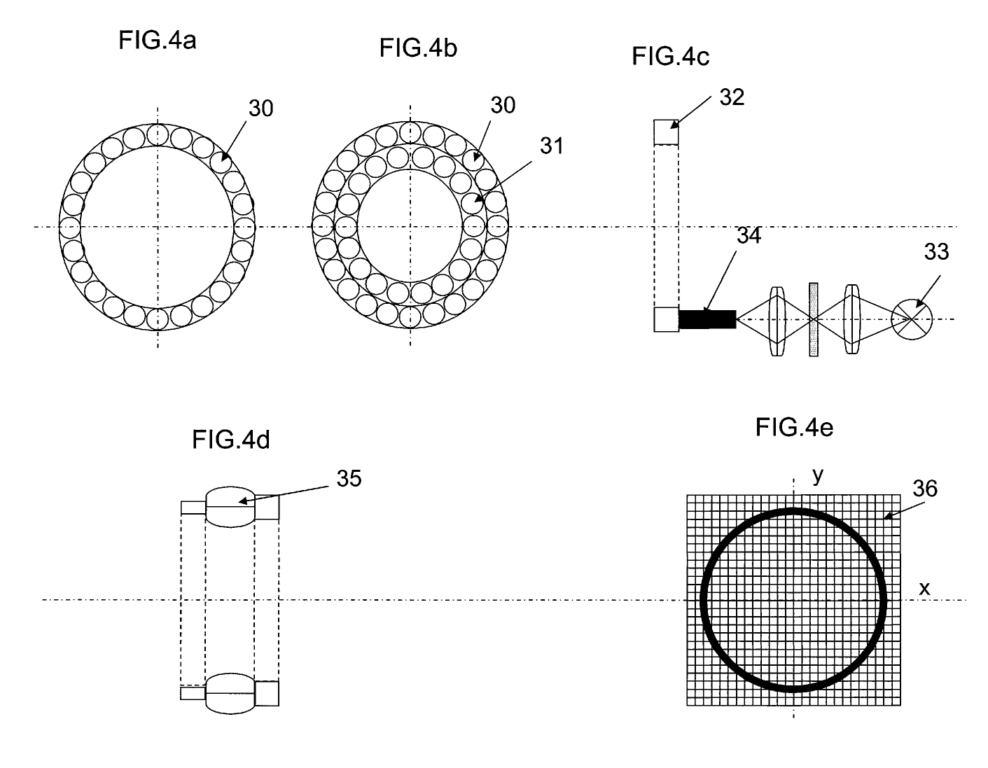

FIGS. 4a through 4e show several exemplary alternative configurations of the ring light 1. FIG. 4a shows a ring light including a plurality of LEDs 30, each having a constant wavelength and small radiation angle. For example, the LEDs may be white for color fundus images, green, 550 nm for high-contrast black-and-white fundus images (as used herein 550 nm means approximately 550 nm), blue, 490-500 nm as excitation light for fluorescence angiography (as used herein 490-500 nm means approximately 490-500 nm, or IR, 880-920 nm as excitation light for ICG angiography (as used herein 880-920 nm means approximately 880 920). The approximate values extend to values above and below the stated values that differ insubstantially in effect.

FIG. 4b shows a ring light having LEDs 30, 31 having with different wavelengths. The LEDs of different wavelengths can always be arranged alternatingly, or else multi-colored LEDs are used, different examination methods being possible with one arrangement.

FIG. 4 shows optical fibers 32 arranged as a ring. In order to be able to carry out several examination methods, an arrangement is proposed in which the light of a halogen lamp 33 is conducted through appropriate filters and condensers into the optical fiber bundle 34.

FIG. 4d shows a ring light source 35 including a taper made either of glass or of PMMA. The source can be a halogen lamp or several LEDs of different wavelengths.

FIG. 4e shows an LED matrix 36. Due to the matrix arrangement of the illuminating LEDs, it is possible to set different ring diameters. Moreover, elliptical illumination can be generated. Through an evaluation of the fundus image being formed, the ring light can be actuated dynamically in the x and y directions and the ring diameter can be varied.

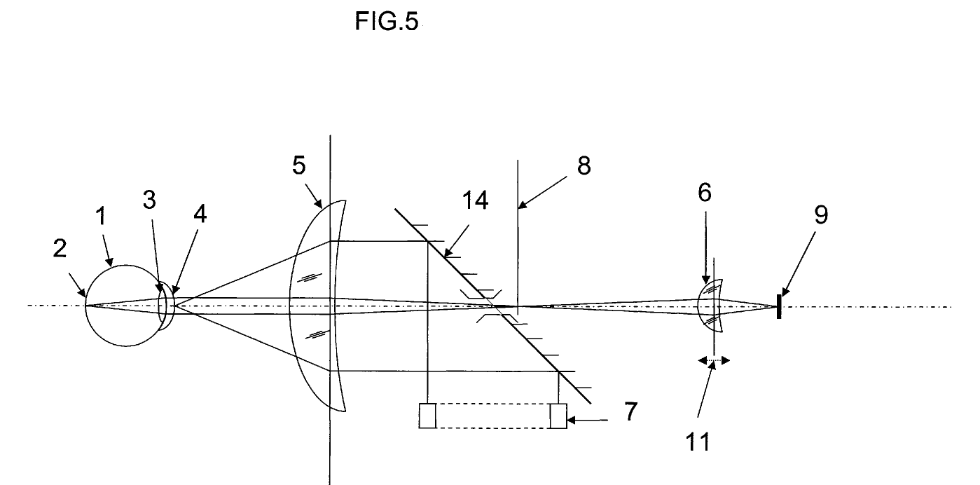

FIG. 5 shows an ophthalmoscope with a solid state camera and ring light via a pinhole mirror. The illumination system is a ring light source 7. The viewing optical path includes a solid state surface sensor 9 located in the imaging plane. The light emitted by the ring light source 7 is assumed to be approximately parallel. The light of the ring light source is reflected in the direction of the ophthalmoscope lens of the main optical axis of the system via a pinhole mirror 14 arranged at 45°. This arrangement has the advantage that it allows greater freedom in terms of the ring light diameter. Moreover, it is conceivable that several ring lights of different diameters and wavelengths can be provided. An LED matrix having very fine structures can also fulfill a ring light function. The ring light is projected via the pinhole mirror 14 and the ophthalmoscope lens 5 onto the cornea 4 of the patient's eye 1. The ring light projected on the cornea 4 scatters light into the inside of the eye 1. The retina 2 constitutes an illuminated object. The eye lens 3 images the retina 2 into infinity and the ophthalmoscope lens 5 focuses it in an intermediate image plane 8. A viewing optical system 6, which in the simplest case comprises an imaging device having an objective with a solid state surface sensor 9, is needed in order to be able to make the intermediate image visible or to capture it. Any unsharpness in the image is compensated for in the direction of the main optical axis by a fine focusing drive 11 of the viewing optical system, or imaging device 6.

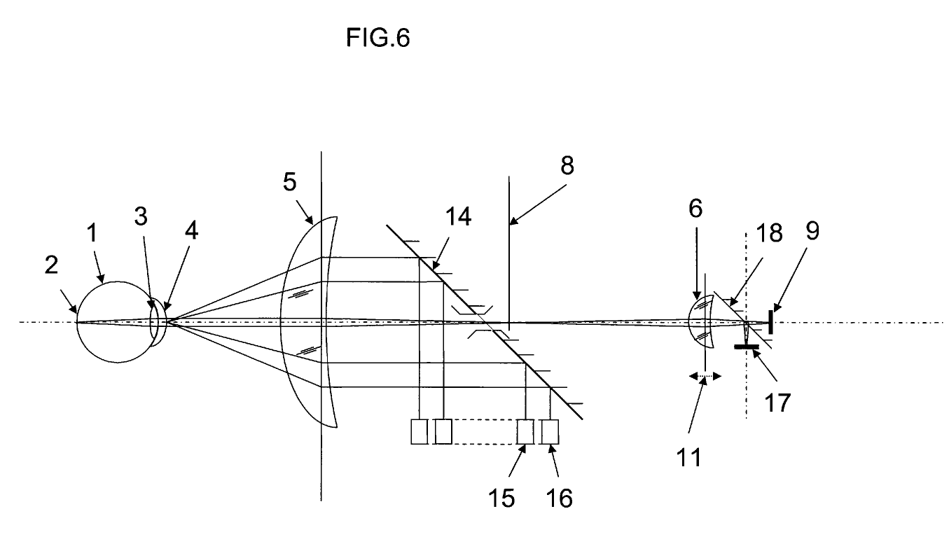

FIG. 6 shows an ophthalmoscope with solid state camera ring light via a pinhole mirror, in a non-mydriatic arrangement. The illumination system is a split ring light source 7. Either every other LED radiates at the same wavelength or else two light rings (as in FIG. 4b) are provided. The light emitted by the ring light source 15 or 16 is assumed to be approximately parallel. The ring light source is reflected into the imaging optical system via a pinhole mirror 14 arranged at 45°. Two ring light arrangements are proposed, IR-LEDs 15 and white LEDs 16. The non-dilated eye of the patient fundamentally reacts to visible light. Illuminating the fundus of the eye with infrared light allows a preliminary examination of the retina. Unfortunately, the images formed do not have a high contrast and are only possible in black-and-white; color images can be taken with a flash in the visible spectrum since the iris only contracts after the flash is over. This method is generally known. According to the invention, the ring light arrangement is divided, with the white LEDs 16 only functioning in flash operation and the IR-LEDs 15 serving for a preliminary examination of the fundus of the eye. The ring light is projected onto the cornea 4 of the patient's eye 1 via the pinhole mirror 14 arranged at 45° and through the ophthalmoscope lens 5. The ring light projected on the cornea 4 scatters light into the inside of the eye 1. The retina 2 constitutes an illuminated object. The eye lens 3 images the retina 2 into infinity and the ophthalmoscope lens 5 focuses it in an intermediate image plane 8. A viewing optical system 6, which in the simplest case comprises an imaging device with an objective with a solid state surface sensor 9, is needed in order to be able to make the intermediate image visible or to capture it. Any unsharpness in the image is compensated for in the direction of the main optical axis by a fine focusing drive 11 of the viewing optical system or imaging device 6. Two solid state cameras are provided, an IR-sensitive camera 17 serving for the preliminary examination, and a color camera 9 (e.g. re-start camera synchronous to the flash) serving to photograph the fundus of the eye. Here, the cameras can be coupled into the viewing optical path either via a partially transparent mirror 18 or via a hinged mirror 18 that briefly swings out when the snapshot is made.

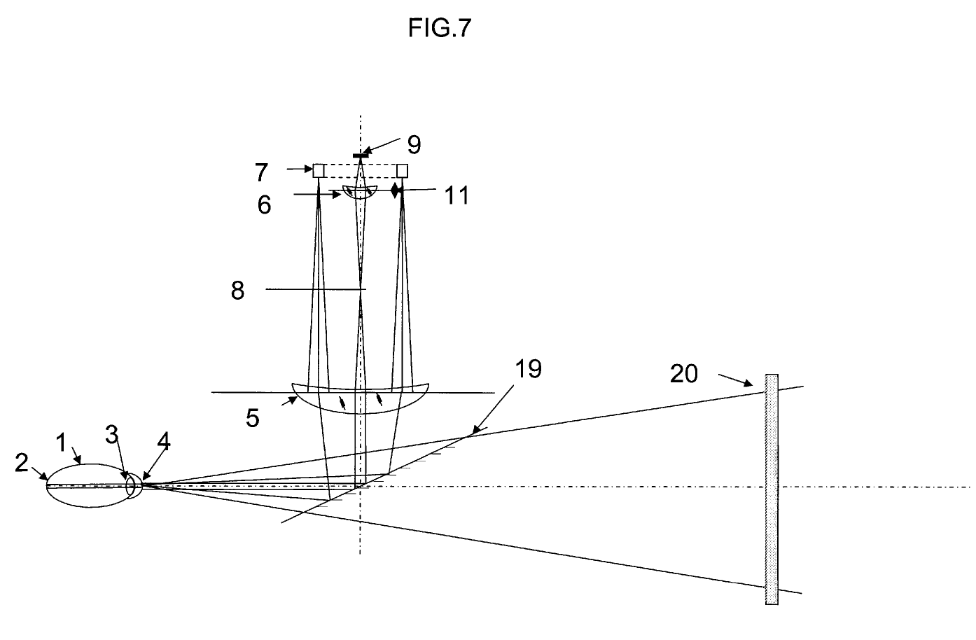

FIG. 7 shows an IR ophthalmoscope with an optical path angled relative to the eye and solid state camera. The illumination system is a ring light source 7. The viewing optical path consists of an IR-sensitive solid state surface sensor 9 located in the imaging plane and having a viewing optical system or imaging device 6. The two optical paths are identical, the ophthalmoscope lens 5 being shared. The light emitted by the IR ring light source 7 is assumed to be approximately parallel. The ring light is projected onto the cornea 4 of the patient's eye 1 through the ophthalmoscope lens 5 of the IR-blocking filter 19 which, at the same time, reflects the infrared light almost completely. The ring light projected on the cornea 4 scatters the IR light into the inside of the eye 1. The retina 2 constitutes an illuminated object. The eye lens 3 images the retina 2 into infinity and the ophthalmoscope lens 5 focuses it in an intermediate image plane 8. A viewing optical system 6, which in the simplest case comprises an imaging device with an objective with an IR-sensitive solid state surface sensor 9, is needed in order to be able to make the intermediate image visible or to capture it. Any unsharpness in the image is compensated for in the direction of the main optical axis by a fine focusing drive 11 of the viewing optical system or imaging device 6. The eye of the patient looks through an IR-blocking filter 19 arranged at an angle of 45° with respect to the viewing axis, said IR-blocking filter 19 serving, at the same time, as an IR mirror, that is to say, the IR ophthalmoscope can be used to view the retina without disrupting the view of the patient. This technique can be used in electro-physiological examinations (e.g. ElectroRetinoGram). The patient looks at stimulating patterns, either on a monitor 20 or a light matrix 20, the observer views the retina of the patient and can evaluate its position. Since it is known that low-contrast images are obtained when IR-illumination of the fundus of the eye is used, an on-line reworking of the camera signal is proposed and it is also possible to use false-color technology.

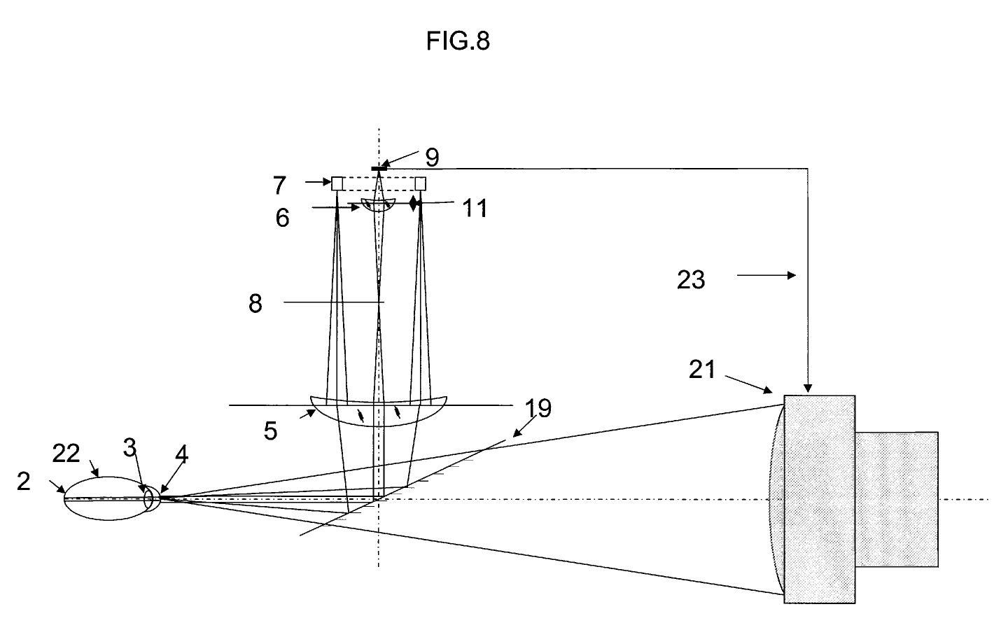

FIG. 8 shows an IR ophthalmoscope with an optical path angled relative to the eye and solid state camera for viewing one's own retina. The illumination system is a ring light source 7. The viewing optical path consists of an IR-sensitive solid state surface sensor 9 located in the imaging plane and having a viewing optical system or imaging device 6. The two optical paths are identical, the ophthalmoscope lens 5 being shared. The light emitted by the IR ring light source 7 is assumed to be approximately parallel. The ring light is projected onto the cornea 4 of the patient's eye 1 through the ophthalmoscope lens 5 of the IR-blocking filter 19 which, at the same time, reflects the infrared light almost completely. The ring light projected on the cornea 4 scatters the IR light into the inside of the eye 1. The retina 2 constitutes an illuminated object. The eye lens 3 images the retina 2 into infinity and the ophthalmoscope lens 5 focuses it in an intermediate image plane 8. A viewing optical system 6, which in the simplest case comprises an imaging device with an objective with an IR-sensitive solid state surface sensor 9, is needed in order to be able to make the intermediate image visible or to capture it. Any unsharpness in the image is compensated for in the direction of the main optical axis by a fine focusing drive 11 of the viewing optical system or imaging device 6. The observer 22 looks at a video monitor 21 through an IR-blocking filter 19 arranged at an angle of 45° with respect to the viewing axis, said IR-blocking filter 19 serving, at the same time, as an IR mirror. The signal 23 of the solid state surface sensor 9 is reproduced in the monitor 21. The observer 22 sees his own retina. Since it is known that low-contrast images are obtained when IR-illumination of the fundus of the eye is used, an on-line reworking of the camera signal is proposed and it is also possible to use false-color technology.

What is claimed is:

1. An ophthalmoscope for viewing a fundus of an eye comprising:
an optical lens;
a ring light source configured to project light along an illumination path through the optical lens into the eye;
a viewing optical system configured to view the fundus through the optical lens along a viewing optical path, wherein a portion of the viewing optical path and a portion of the illuminating optical path share a same optical axis.

2. The ophthalmoscope as recited in claim 1, wherein the viewing optical system includes a solid state camera.

3. The ophthalmoscope as recited in claim 1, wherein the viewing optical system includes an eyepiece.

4. The ophthalmoscope as recited in claim 1, wherein the ring light source defines a diameter and wherein the diameter is variably adjustable.

5. The ophthalmoscope as recited in claim 1, wherein the ring light includes a plurality of LEDs.

6. The ophthalmoscope as recited in claim 5, wherein the LEDs have a constant wavelength and a small radiation angle.

7. The ophthalmoscope as recited in claim 6, wherein the constant wavelength corresponds to at least one of white, green, blue, and infra red.

8. The ophthalmoscope as recited in claim 7, wherein the constant wavelength is one of 550 nm, 490-500 nm and 880-920 nm.

9. The ophthalmoscope as recited in claim 5, wherein the LEDs have different wavelengths.

10. The ophthalmoscope as recited in claim 1, wherein the ring light includes optical fibers arranged on a ring.

11. The ophthalmoscope as recited in claim 1, wherein the ring light includes an LED matrix enabling different ring diameters to be set.

12. The ophthalmoscope as recited in claim 1, further comprising a pinhole mirror, the viewing optical path passing through a pinhole in the pinhole mirror.

13. The ophthalmoscope as recited in claim 12, wherein the ophthalmoscope includes a non-mydriatic arrangement.

14. The ophthalmoscope as recited in claim 1, further comprising an IR blocking filter.

15. The ophthalmoscope as recited in claim 14, further comprising at least one of a monitor and a light matrix.

16. A fundus camera comprising:
a viewing optical path;
an imaging device; and
an illuminating optical path including at least one LED and a pinhole mirror reflecting the at least one LED into the imaging device, wherein at least a portion of the illuminating optical path shares an optical axis with at least a portion of the viewing optical path.

17. The fundus camera as recited in claim 16, wherein the at least one LED includes at least one first LED and at least one second LED, the first and second LEDs having different wavelengths.

18. The fundus camera as recited in claim 16, wherein the at least one LED includes at least one first LED emitting green light, at least one second LED emitting blue light and at least one third LED emitting IR light.

19. The fundus camera as recited in claim 18, wherein the first, second and third LEDs are configured to be actuated dynamically.

20. The fundus camera as recited in claim 17, wherein the first LED emits at 550 mn, the second LED emits at 490-500 nm and the third LED emits at 880-920 nm.

21. The fundus camera as recited in claim 18, wherein the at least one third LEDs is configured for a preliminary examination of an eye.

22. The fundus camera as recited in claim 16, wherein the at least one LED supplies white light.

23. The fundus camera as recited in claim 22, wherein at least one LED is configured for flash operation.

24. The fundus camera as recited in claim 16, wherein the at least one LED includes a plurality of LEDs disposed in a shape of a ring.

25. The fundus camera as recited in claim 24, wherein the ring has a variable diameter.

26. The fundus camera as recited in claim 16, wherein the at least one LED includes a plurality of LEDs disposed in a matrix arrangement.

27. The fundus camera as recited in claim 16, wherein the imaging device includes a solid state camera.

28. The fundus camera as recited in claim 27, wherein the solid state camera operates synchronously to the at least one LED in flash operation.

29. The fundus camera as recited in claim 27, wherein the imaging device includes two solid state cameras configured to be coupled into the viewing optical path via a mirror.

30. The fundus camera as recited in claim 29, wherein the mirror is one of a partially transparent mirror and a moveable hinged mirror.

31. The fundus camera as recited in claim 16, wherein the illuminating optical path includes an optical fiber bundle.

32. The fundus camera as recited in claim 16, wherein the illuminating optical path has a taper.

33. The fundus camera as recited in claim 32, wherein the taper is made of at least one of glass and PMMA.

34. The fundus camera as recited in claim 16, further comprising an illuminated object, wherein the illuminated object includes a retina, wherein the at least one LED includes a plurality of LEDs emitting light having a constant wavelength and a small radiation angle, wherein the LEDs are configured to be actuated individually and projected onto the cornea of the eye.

35. The fundus camera as recited in claim 16, wherein the fundus camera includes a non-mydriatic arrangement.
